SEQUENCE LISTING (4 sequences)

SEQ ID NO: 1 - DNA, 22 bp, Artificial Sequence
  IL-6 F primer
gtccttcctaccccaatttcca

SEQ ID NO: 2 - DNA, 20 bp, Artificial Sequence
  IL-6 R primer
taacgcactaggtttgccga

SEQ ID NO: 3 - DNA, 20 bp, Artificial Sequence
  GAPDH F primer
ctcactcaagattgtcagca

SEQ ID NO: 4 - DNA, 20 bp, Artificial Sequence
  GAPDH R primer
gtcatcatacttggcaggtt

(19) United States
(12) Patent Application Publication
(10) Pub. No.: US 2023/0277594 A1
(43) Pub. Date: Sep. 7, 2023

(54) METHOD FOR DIFFERENTIATING MESENCHYMAL STEM CELLS FROM PLURIPOTENT STEM CELLS

(71) Applicant: KONKUK UNIVERSITY INDUSTRIAL COOPERATION CORP, Seoul (KR)

(72) Inventors: Ssang-Goo CHO, Seoul (KR); Ahmed ABDAL DAYEM, Seoul (KR); Soo Bin LEE, Seoul (KR)

(73) Assignee: KONKUK UNIVERSITY INDUSTRIAL COOPERATION CORP, Seoul (KR)

(21) Appl. No.: 17/997,988

(22) PCT Filed: May 7, 2021
(86) PCT No.: PCT/KR2021/005767

§ 371 Date: Nov. 4, 2022

(30) Foreign Application Priority Data
May 7, 2020 (KR) 10-2020-0054455

(51) Int. Cl. A61K 35/28; C12N 5/0775; A61P 29/00

(52) U.S. Cl. CPC A61K 35/28; C12N 5/0668; A61P 29/00; C12N 2502/45; C12N 2533/54; C12N 2513/00; C12N 2525/00

(57) ABSTRACT

The present disclosure relates to a method for producing mesenchymal stem cells from pluripotent stem cells and mesenchymal stem cells prepared by the method. The present disclosure enables mesenchymal stem cells having superior proliferation rate while having superior intrinsic biological activity to be obtained with high yield by sequentially performing three-dimensional suspension culture using pluripotent stem cells, particularly induced pluripotent stem cells (iPSCs), as starting cells and adherent culture of cell aggregates formed therethrough. The mesenchymal stem cells produced by the method of the present disclosure can be usefully used in compositions for treating bone diseases, cartilage diseases or inflammatory and autoimmune diseases owing to high differentiation efficiency into bone and cartilage and superior anti-inflammatory activity.

### TECHNICAL FIELD

The present disclosure relates to a method for differentiating mesenchymal stem cells from pluripotent stem cells by sequentially performing three-dimensional culture and adherent culture under microgravity.

### BACKGROUND ART

Human pluripotent stem cells (hPSCs) including human embryonic stem cells (hESCs) and induced pluripotent stem cells (iPSCs) can propagate indefinitely while retaining their ability to differentiate into various types of somatic cells. These cells valued highly because they provide unlimited cell source in cell therapy and regenerative medicine, and researches are conducted actively on various methods for inducing differentiation into specific cell types suitable for the tissue to be regenerated, methods for isolating the differentiated cells and methods for removing undifferentiated cells.

Mesenchymal stem cells (MSCs), which were first identified in the bone marrow, are pluripotent cells with a great potential in regenerative medicine. The mesenchymal stem cells can differentiate into various cell types of the mesenchymal lineage, such as bone cells, cartilage cells, fat cells, muscle cells, fibroblasts, etc. With immunoregulatory activity, they can be used as graft enhancers and in compositions for treating various autoimmune and inflammatory diseases such as fetal graft-versus-host disease, etc. (Le Blanc K et al., Lancet, 363(9419): 1439-1441, 2004; El-Badri N. S et al., Exp Hematol, 26(2): 110-116, 1998). The mesenchymal stem cells can be isolated from various human tissues such as the bone marrow, adipose tissue, umbilical cord blood, peripheral blood, neonatal tissue, placenta, etc. However, the number of mesenchymal stem cells that can be obtained from the adult tissues is limited and the invasive procedure required to isolate the mesenchymal stem cells may unexpectedly harm the donor. Therefore, the inventors of the present disclosure have aimed at presenting a new alternative for acquiring therapeutic stem cells in regenerative medicine by developing a method for effectively obtaining a therapeutically effective amount of mesenchymal stem cells from pluripotent stem cells.

Throughout the present specification, a number of publications and patent documents are referred to and cited. The disclosure of the cited publications and patent documents is incorporated herein by reference in its entirety to more clearly describe the state of the related art and the present disclosure.

### REFERENCES OF RELATED ART

### Patent Documents

(Patent document 1) Patent document 1. Korean Patent Application No. 10-2011-0107237.

### DISCLOSURE

### Technical Problem

The inventors of the present disclosure have made consistent efforts to develop a method for effectively differentiating mesenchymal stem cells having excellent clinical utility from pluripotent stem cells. As a result, they have found out that mesenchymal stem cells having superior intrinsic pharmacological effect such as tissue-regenerating and immunoregulatory activities and having excellent proliferation rate can be obtained with high yield by forming spheroids by culturing embryoid bodies obtained from suspension culture of pluripotent stem cells in a three-dimensional bioreactor under artificially induced zero gravity or microgravity and then adherent-culturing them in a culture vessel coated with an adhesive polymer, and have completed the present disclosure.

The present disclosure is directed to providing a method for producing mesenchymal stem cells from pluripotent stem cells.

The present disclosure is also directed to providing mesenchymal stem cells produced by the method of the present disclosure and a composition for treating a bone disease, a cartilage disease, an inflammatory disease or an autoimmune disease, which contains the same as an active ingredient.

Other purposes and advantages of the present disclosure will become more apparent by the following detailed description, claims and drawings.

### Technical Solution

In an aspect, the present disclosure provides a method for producing mesenchymal stem cells from pluripotent stem cells, which includes:
(a) a step of forming embryoid bodies (EBs) by culturing pluripotent stem cells isolated from a subject;
(b) a step of forming spheroids by three-dimensionally culturing the embryoid bodies in a bioreactor under microgravity; and
(c) a step of differentiating the spheroids into mesenchymal stem cells by adherent-culturing in a culture vessel coated with an adhesive polymer.

The inventors of the present disclosure have made consistent efforts to develop a method for effectively differentiating mesenchymal stem cells having excellent clinical utility from pluripotent stem cells. As a result, they have found out that mesenchymal stem cells having superior intrinsic pharmacological effect such as tissue-regenerating and immunoregulatory activities and having excellent proliferation rate can be obtained with high yield by forming spheroids by culturing embryoid bodies obtained from suspension culture of pluripotent stem cells in a three-dimensional bioreactor under artificially induced zero gravity or microgravity and then adherent-culturing them in a culture vessel coated with an adhesive polymer.

In the present specification, the term “stem cells” collectively refer to undifferentiated cells prior to differentiation into individual cells constituting tissues, which have the ability to differentiate into specific cells under specific stimulation (environment). Unlike differentiated cells whose cell division has stopped, the stem cells retain the capability of self-renewal through cell division and have the plasticity of differentiation to differentiate into various cells under different stimuli.

The stem cells used in the present disclosure may be any cells that have the characteristics of stem cells, i.e., being undifferentiated, unlimited proliferation and ability to differentiate into specific cells, and thus can be induced to differentiate into the tissue desired to be regenerated, without limitation.

In a specific exemplary embodiment of the present disclosure, the stem cells used in the present disclosure are mesenchymal stem cells.

In the present specification, the term “mesenchymal stem cells” refer to stem cells having the multipotency of differentiating into fat cells, bone cells, cartilage cells, muscle cells, nerve cells and cardiac muscle cells. The mesenchymal stem cells can be distinguished by their spiral shapes and the expression level of the basic cell surface markers CD73(+), CD105(+), CD34(−) and CD45(−). They have the function of regulating immune response in addition to the multipotency.

In the present specification, the term “pluripotent stem cells” refers to stem cells that have developed from fertilized eggs and can differentiate into all cells constituting the endoderm, mesoderm and ectoderm. In a specific exemplary embodiment of the present disclosure, the pluripotent stem cells used in the present disclosure are embryonic stem cells (ESCs), embryonic germ cells, embryonic carcinoma cells or induced pluripotent stem cells (iPSCs), more specifically embryonic stem cells or induced pluripotent stem cells, most specifically induced pluripotent stem cells.

In the present specification, the term “induced pluripotent stem cells” refers to pluripotent stem cells artificially derived from non-pluripotent cells (e.g., somatic cells) by insertion of a specific gene. The induced pluripotent stem cells are considered as having the same phenotypical, physiological and embryological characteristics as natural pluripotent stem cells such as embryonic stem cells, in many aspects, such as expression stem cell genes and proteins, chromatin methylation, doubling time, embryoid body formation, teratoma formation, viable chimera formation, hybridizability and differentiability.

In the present specification, the term “differentiation of stem cells” includes not only the induction of complete differentiation of undifferentiated stem cells into specific cells but also the formation of precursor cells before differentiation from stem cells to specific cells.

The cell culture used in each step of the present disclosure is a mixture for growth and proliferation of cells in vitro, which contains ingredients essential for the growth and proliferation of cells such as sugars, amino acids, various nutrients, minerals, etc. The ingredients that can be further contained in a cell culture medium include, for example, glycerin, L-alanine, L-arginine hydrochloride, L-cysteine hydrochloride monohydrate, L-glutamine, L-histidine hydrochloride monohydrate, L-lysine hydrochloride, L-methionine, L-proline, L-serine, L-threonine, L-valine, L-asparagine monohydrate, L-aspartic acid, L-cystine 2HCl, L-glutamic acid, L-isoleucine, L-leucine, L-phenylalanine, L-tryptophan, L-tyrosine disodium salt dihydrate, i-inositol, thiamine hydrochloride, niacinamide, pyridoxine hydrochloride, biotin, calcium D-panthothenate, folic acid, riboflavin, vitamin B₁₂, sodium chloride (NaCl), sodium bicarbonate (NaHCO₃), potassium chloride (KCl), calcium chloride (CaCl₂), sodium dihydrogen phosphate monohydrate (NaH₂PO₄·H₂O), copper sulfate pentahydrate (CuSO₄·5H₂O), iron(II) sulfate heptahydrate (FeSO₄·7H₂O), magnesium chloride (anhydrous), magnesium sulfate (MgSO₄), disodium hydrogen phosphate (Na₂HPO₄), zinc sulfate heptahydrate (ZnSO₄·7H₂O), D-glucose (dextrose), sodium pyruvate, hypoxanthine Na, linolenic acid, lipoic acid, putrescine 2HCl and thymidine, although not being limited thereto.

The cell culture medium according to the present disclosure may be prepared artificially or may be purchased from commercially available ones. Examples of the commercially available culture medium include IMDM (Iscove's modified Dulbecco's medium), α-MEM (alpha modification of Eagle's medium), F12 (nutrient mixture F-12) and DMEM/F12 (Dulbecco's modified Eagle's medium: nutrient mixture F-12), although not being limited thereto.

In a specific exemplary embodiment of the present disclosure, the step (a) is performed by three-dimensionally culturing the pluripotent stem cells in a multi-well culture plate.

In the present specification, the term “three-dimensional culture” is a concept opposite to two-dimensional culture, and refers to culturing of cells in a floating state in a culture medium without adhesion to a substrate, etc. Thus, the term “three-dimensional culture” is used in the same meaning as “suspension culture”. Adhesion-dependent stem cells tend to aggregate during suspension culture. Because the cells that float without participating in the aggregation die of apoptosis, an environment corresponding to the adhesion property should be created. According to the present disclosure, by suspension-culturing pluripotent stem cells in a multi-well plate having a plurality of wells, pluripotent cell aggregates, i.e., embryoid bodies (EBs), of different sizes can be formed in the wells of different sizes (diameters). Therefore, standardized embryoid bodies having identical size and shape can be obtained in large quantities in the step (a) of the present disclosure.

More specifically, the multi-well culture plate is a microwell plate having wells with a size of 350 μm×350 μm to 450 μm×450 μm.

In a specific exemplary embodiment of the present disclosure, the suspension culture is performed by seeding 0.5×10⁵ to 1.5×10⁵ cells per well of the multi-well culture plate. More specifically 0.7×10⁵ to 1.3×10⁵ cells, most specifically 0.9×10⁵ to 1.1×10⁵ cells are seeded.

More specifically, the step (a) further includes a step of inducing cell aggregation during the suspension culture through centrifugation.

In the present specification, the term “cell aggregate” refers to a cell mass of a three-dimensional structure formed as cells cultured in an environment allowing three-dimensional growth, such as suspension culture, etc., rather than monolayer growth are self-aggregated. The cell aggregate obtained through three-dimensional culturing provides an environment similar to that of the tissue from which the stem cells are derived. Depending on the size and the number of the self-aggregated cells, it may have a spherical or other shape. The cell aggregate having a spherical shape is called a spheroid.

In a specific exemplary embodiment of the present disclosure, the microgravity in the step (b) is induced by a microgravity simulator which offsets the gravity applied to the bioreactor by rotating the bioreactor.

In the present specification, the term “microgravity” refers to zero gravity, undetectably small gravity or gravity which does not affect detectable biological or physiological effect. Specifically, it refers to an environment of 1×10⁶ g or lower. The term “microgravity” may also be expressed as “weightlessness”.

In the present specification, the term “microgravity simulator” refers to a device which induces a microgravity environment by artificially offsetting in an environment of normal or significant gravity. The microgravity simulator may be, for example, a clinostat, a random positioning machine (RPM), a rotating wall vessel (RWV), etc. However, any device capable of offsetting gravity in the culture environment in the bioreactor the present disclosure for a predetermined time by applying appropriate external force may be used without being limited thereto.

In a specific exemplary embodiment of the present disclosure, the microgravity simulator of the present disclosure is a clinostat. A clinostat is a device which is coupled to a culture vessel such as a bioreactor and offsets gravity by rotating continuously while changing directions randomly or according to a preset (input) pattern.

In a specific exemplary embodiment of the present disclosure, the step (b) is performed by culturing the embryoid bodies for 3-8 days, more specifically for 4-7 days, most specifically for 5 days, while rotating the microgravity simulator at 40-80 rpm.

More specifically, the step (b) is performed by rotating the microgravity simulator first at 40-60 rpm and increasing the rotation speed by 5 rpm every day. Specifically, the embryoid bodies are cultured for 5 days by rotating the microgravity simulator first at 50 rpm and increasing the rotation speed by 5 rpm every day.

In the present specification, the term “bioreactor” refers to a culture apparatus or system for a biological sample, which includes a culture space for creating a culture environment with biological activity and a series of machinery operating interactively therewith.

According to the present disclosure, in the step (b) of the method of the present disclosure, spheroids are formed by three-dimensionally culturing the embryoid bodies produced in the step (a) in a bioreactor under microgravity. The spheroids refer to cell aggregates with spherical shapes, but they need not to be perfectly spherical geometrically.

According to the present disclosure, after the two three-dimensional suspension cultures in the step (a) and the step (b), the formed spheroids are differentiated into mesenchymal stem cells by adherent-culturing in a culture vessel coated with an adhesive polymer.

In the present specification, the term “polymer” refers to a synthetic or natural polymer compound wherein identical or different monomers are bonded continuously. Accordingly, the polymer includes a homopolymer (a polymer polymerized from one monomer) and a copolymer prepared from polymerization of at least two different monomers. The copolymer includes a bipolymer (a polymer prepared from polymerization of two different monomers) and a polymer prepared from more than two different monomers.

In the present specification, the term “adhesive polymer” refers to a natural or artificial polymer, which forms crosslinkage between the culture surface and cells or cell aggregates (e.g., spheroids) through covalent or non-covalent bonding and, thus, allows the aggregates to be cultured without being detached from the bottom or sidewall of the culture vessel, i.e. in the adhered state.

In a specific exemplary embodiment of the present disclosure, the adhesive polymer is selected from a group consisting of hyaluronic acid, alginate, heparin, fucoidan, cellulose, dextran, chitosan, albumin, fibrin, collagen and gelatin. More specifically, the adhesive polymer is gelatin.

In another aspect, the present disclosure provides mesenchymal stem cells produced by the method of the present disclosure.

In another aspect, the present disclosure provides a composition for treating a bone or cartilage disease, which contains the mesenchymal stem cells of the present disclosure as an active ingredient.

In the present specification, the term “treatment” refers to (a) suppressing the development of a disorder, a disease or symptoms; (b) reducing the disorder, disease or symptoms; or (c) removing the disorder, disease or symptoms. The mesenchymal stem cells differentiated by the method of the present disclosure effectively differentiate into bone and cartilage and, thereby, suppress, remove or alleviate the symptoms of various bone or cartilage diseases caused by irreversible loss of bone or cartilage tissues. Accordingly, the composition of the present disclosure may be used as a composition for treating a disease on its own, or may be used as a therapeutic adjuvant for the disease when administered with in combination with another pharmaceutical ingredient having therapeutic effect for the inflammatory or autoimmune disease. Therefore, in the present specification, the term “treatment” or “therapeutic agent” encompasses “assistance of treatment” or “therapeutic adjuvant”.

In the present specification, the term “administration” refers to direct administration of a therapeutically effective amount of the composition of the present disclosure to a subject, so that the same amount is formed in the body of the subject. It has the same meaning as “transplantation” or “injection”. In the present specification, the term “transplantation” refers to a process of delivering living cells from a donor or an artificial scaffold, etc. supporting the same to a recipient in order to maintain the functional integrity of the transplanted cells in the recipient.

In the present specification, the term “therapeutically effective amount” refers to the amount of the composition of the present disclosure which is sufficient to provide a therapeutic or prophylactic effect in a subject. Therefore, the term encompasses “prophylactically effective amount”.

In the present specification, the term “subject” includes human, mouse, rat, guinea pig, dog, cat, horse, cow, pig, monkey, chimpanzee, baboon or rhesus monkey without limitation. Specifically, the subject of the present disclosure is human.

In the present specification, the term “bone disease” includes all diseases accompanied by or having the risk of being accompanied by bone tissue damage induced by various causes including trauma, bone fracture, bone metastasis of cancer cells, hyperactivity of osteoclasts, etc. Specifically, the bone disease that can be prevented or treated with the composition of the present disclosure includes, for example, osteoporosis, osteogenesis imperfecta, periodontal disease, bone fracture, metabolic osteitis, fibrous osteitis, aplastic bone disease, osteomalacia, rickets, hypercalcemia, multiple myeloma and Paget's disease, although not being limited thereto.

In the present specification, the term “cartilage disease” refers to a disease in which the cartilage tissue loses its intrinsic function due to the death of cartilage cells or decrease of cartilage tissue, such as degenerative arthritis.

In another aspect, the present disclosure provides a composition for treating an inflammatory or autoimmune disease, which contains the mesenchymal stem cells of the present disclosure as an active ingredient.

According to the present disclosure, the mesenchymal stem cells differentiated by the method of the present disclosure remarkably inhibit inflammatory factors in cells wherein inflammation is induced with LPS and thus have superior anti-inflammatory and immunoregulatory activities.

In a specific exemplary embodiment of the present disclosure, the inflammatory or autoimmune disease prevented or treated by the composition of the present disclosure includes, for example, rheumatoid arthritis, reactive arthritis, type 1 diabetes, type 2 diabetes, systemic lupus erythematosus, multiple sclerosis, cryptogenic fibrosing alveolitis, polymyositis, dermatomyositis, localized scleroderma, systemic scleroderma, colitis, inflammatory bowel disease, Sjorgen's syndrome, Raynaud's phenomenon, Bechet's disease, Kawasaki's disease, primary biliary sclerosis, primary sclerosing cholangitis, ulcerative colitis, graft-versus-host disease (GVHD) and Crohn's disease, although not being limited thereto.

When the composition of the present disclosure is prepared as a pharmaceutical composition, the pharmaceutical composition of the present disclosure contains a pharmaceutically acceptable carrier. The pharmaceutical composition may be formulated into a single-dosage form suitable for administration into the body of a patient according a method common in the art. Parenteral formulations suitable for this purpose include a solution or suspension for injection, an ointment as a formulation for topical administration, etc. The composition of the present disclosure may be formulated using a commonly used diluent or excipient such as a filler, an extender, a binder, a wetting agent, a disintegrant, a surfactant, etc. The administration dosage of the composition may be about 1 μg to 50 mg per kg body weight, and the administration dosage of the therapeutic stem cells for an adult may be 1 to 10⁸, 10 to 10⁵ or 10² 10³ cells per day. The administration may be made once or several times a day.

However, the dosage and number of the administration may be determined in consideration of not only the severity of a disease and administration route but also such factors as the body weight, sex, etc. of a patient.

### Advantageous Effects

The features and advantages of the present disclosure may be summarized as follows:
(a) The present disclosure provides a method for producing mesenchymal stem cells from pluripotent stem cells and mesenchymal stem cells produced by the method.
(b) According to the present disclosure, mesenchymal stem cells having superior intrinsic biological activity and excellent proliferation rate can be obtained with high yield by sequentially performing three-dimensional suspension culture using pluripotent stem cells, particularly induced pluripotent stem cells (iPSCs), as starting cells and adherent culture of cell aggregates formed therethrough.
(c) The mesenchymal stem cells produced by the method of the present disclosure can be usefully used in compositions for treating bone diseases, cartilage diseases or inflammatory and autoimmune diseases owing to high differentiation efficiency into bone and cartilage and superior anti-inflammatory activity.

### BRIEF DESCRIPTION OF DRAWINGS

FIG. 1 schematically illustrates a protocol of the present disclosure whereby MSCs are differentiated from iPSCs.

FIG. 2 shows the images of embryoid bodies (EBs) formed on AggreWell.

FIG. 3 shows spheroids formed using the microgravity bioreactor BAM (Bio Array Matrix) (top) and a result of staining them with OCT4 and DAPI antibodies (bottom).

FIG. 4 shows the morphology of mesenchymal stem cells derived from spheroids. It can be seen that the cells exhibit a spindle shape after passaging (right).

FIG. 5 shows the morphology of MSCs differentiated by a method of the present disclosure depending on passages.

FIGS. 6a-6c show the cumulative cell proliferation of mesenchymal stem cells differentiated by a method of the present disclosure. Cumulative population doubling (CPD) (FIG. 6a), doubling time (FIG. 6b) and logarithmic cell number (FIG. 6c) depending on passages are shown.

FIG. 7 shows a result of confirming the expression of cell surface markers in mesenchymal stem cells differentiated by a method of the present disclosure by FACS analysis.

FIG. 8 shows a result of confirming that mesenchymal stem cells differentiated by a method of the present disclosure differentiated into fat, bone and cartilage through Alizarin Red S, Oil Red O and Alcian blue staining.

FIG. 9 shows a result of confirming that pluripotent stem cells induced by a method of the present disclosure lose pluripotency and differentiate into cells expressing the markers of mesenchymal stem cells by immunocytochemistry.

FIG. 10 schematically illustrates an experimental procedure of confirming the anti-inflammatory effect of stem cells differentiated from LPS-induced inflammatory cells by a method of the present disclosure.

FIG. 11 shows a result of confirming the expression of inflammatory markers by RT-PCR.

### BEST MODE

Hereinafter, the present disclosure will be described in more detail through examples. The examples are only for describing he present disclosure specifically, and it will be obvious to those having ordinary knowledge in the art that the scope of the present disclosure is not limited by the examples.

### Examples

### Experimental Method

Culturing of Single Colonies

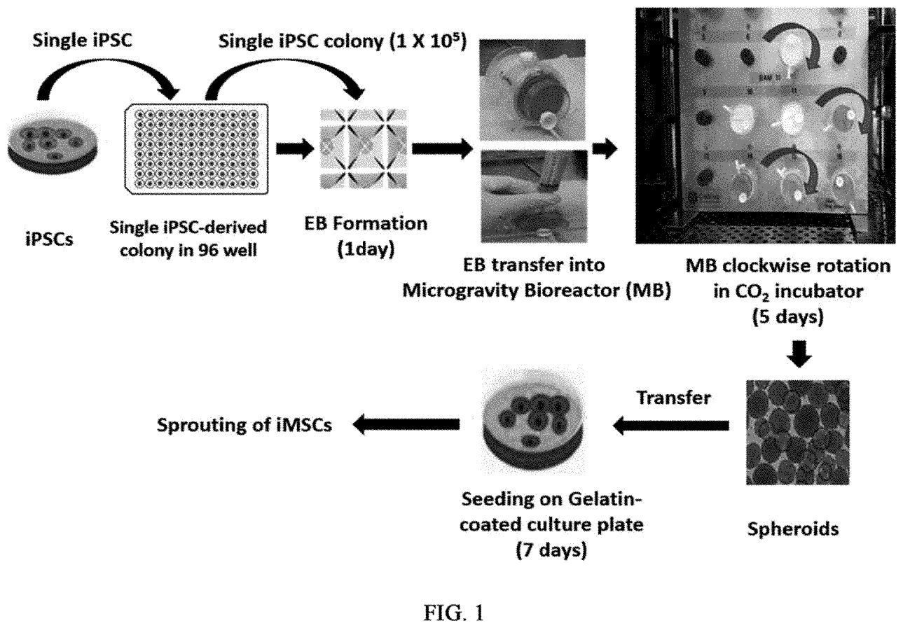

Single colonies were formed by culturing single iPSCs adhered onto a Matrigel (354234, Corning, USA)-coated 96-well plate for a week using an iPSC medium. Each colony was passaged using Matrigel-coated 24-well dish and 6-well dish, subsequently. When the cell number was increased to about 1×10⁵, the cells were used for spheroid experiments (FIG. 1).

Formation of Spheroids Using BAM System

After seeding about 1×10⁵ iPSCs onto an AggreWell plate (34460, StemCell, Canada), the cells were aggregated by centrifuging at 300 g for 5 minutes and then cultured in a 5% CO₂ incubator for 24 hours to form embryoid bodies (EBs). The EBs were cautiously transferred into a bioreactor (CelVivo, Denmark) and rotated for 5 days using the microgravity device BAM (CelVivo, Denmark). The rotation was started at 50 rpm and the rotation speed was increased by 5 rpm every day (FIG. 1).

Production of iPSC-MSCs

The spheroids grown in the BAM system were transferred to a 0.1% gelatin-coated 6-well culture dish and cultured using DMEM/F12 supplemented with 10% FBS and 1% P/S while replacing the medium every 2-3 days. When the cells grew from the spheroids attached to the coated surface to 70-80% confluency, they were passaged using TrypLE. The first passage was denoted as P0, and the passaging was continued until homogenous cell morphology was achieved.

Investigation of Pluripotency of Mesenchymal Stem Cells by Immunohistochemical (ICC) Staining

The spheroids formed from the iPSCs were taken out of the bioreactor and fixed with 4% paraformaldehyde (PFA). 1×10⁵ iPSC-MSCs formed from the spheroids of the iPSCs were seeded onto a confocal dish (101350, SPL, Korea) and then fixed with 4% PFA at 60-70% confluency. The fixed spheroids and iPSC-MSCs were washed 3 times with DPBS for 5 minutes and the surface was permeabilized by treating with 0.3% Triton X-100. Then, after washing 3 times with DPBS for 5 minutes, the washed spheroids were cultured with 3% BSA/PBS at room temperature for 1 hour for blocking. After removing 3% BSA/PSB, the spheroids were reacted with the primary antibodies (1:200) anti-OCT4, anti-SSEA4 and anti-PDGFRβ for 12 hours in a refrigerator. Then, the spheroids were taken out and washed 3 times with DPBS for 5 minutes. After the washing, the spheroids were incubated with the secondary antibody (1:200) goat anti-mouse 488 at room temperature for 1 hour. Then, after removing the secondary antibody and staining the nuclei with DAPI or Topro3 for 20 minutes, the spheroids were washed 3 times with DPBS for 5 minutes. The washed sample was stored in an antifade mounting medium (H-1000, Vector Laboratory, UK) to prevent loss of fluorescence.

Passaging of iPSC-MSCs and Cell Growth Curves

When the morphology of the iPSC-MSCs became homogenous, the cell number was counted to create a growth curve. After seeding 2×10⁵ iPSC-MSCs onto a 60-mm culture dish from P5, the cells were cultured for 5 days while replacing the medium once in 5 days. The cell proliferation was investigated with three growth curves. First, cumulative population doubling (CPD) was calculated by “CPD=log(number of harvested cells/number of seeded cells)/log(2)”. Then, doubling time, i.e., the time spent until the cell number was doubled, was calculated by “doubling time=days of culturing×log(2)/(log(number of harvested cells)−log(number of seeded cells))”. And, the logarithm of the number of passaged cells was calculated by “cell number=log(number of cells)”. The calculated values were plotted as graphs. AD-MSCs and hWJ-MSCs were used as control groups.

Immunophenotype Testing by Flow Cytometry Analysis (FACS)

After detaching cells during culturing using TrypLE™ Express (10624013, GIBCO, USA) and centrifuging at 1,500 rpm for 5 minutes, the supernatant was removed and suspended in a FACS buffer (D-PBS supplemented with 2% FBS), and then reacted primarily with mouse anti-CD34, mouse anti-CD45, mouse anti-CD73 and sheep anti-CD90. The cells were incubated at 4° C. for 30 minutes after adding 200 μL of these primary antibodies diluted to 1:500. Then, after washing with D-PBS and centrifuging at 1,500 rpm for 5 minutes, the supernatant was removed and incubated at 4° C. for 20 minutes after adding 200 μL of rabbit anti-mouse 488 or donkey anti-sheep PE, as a secondary antibody, diluted to 1:500. Then, the stained cells were subjected to flow cytometry analysis (FACS Calibur; Becton Dickinson, Heidelberg, Germany) by suspending in 500 μL of a FACS buffer, by using the Cell Quest pro software.

Induction of Differentiation into Bone, Fat and Cartilage

In order to induce differentiation into three lineages, the cells were adhered onto a 24-well plate at 2×10⁴ cells/well and differentiation was started when the density reached 80%.

For osteogenic differentiation, 10% FBS, 1% penicillin/streptomycin (P/S), 100 nM dexamethasone (Sigma-Aldrich, MO, USA), 50 μg/mL ascorbate-2-phosphate (Sigma-Aldrich, MO, USA) and 10 mM β-glycerophosphate (Sigma-Aldrich, MO, USA) were added to DMEM (Dulbecco's modified Eagle's medium)-low glucose (Invitrogen, CA, USA). The differentiation medium was replaced every 2 days for 2 weeks. When the differentiation was completed, the cells were fixed with 4% PFA for 15 minutes and then washed with sterile water. The differentiation was confirmed by staining calcium phosphate deposits with Alizarin Red S.

For adipocyte differentiation, 10% FBS, 1% P/S, 500 μM isobutylmethylxanthine, 1 μM dexamethasone, 100 μM indomethacin and 10 μg/mL insulin were added to DMEM-high glucose. The differentiation medium was replaced every 3 days for 2 weeks. When the differentiation was completed, the cells were fixed with 4% PFA for 15 minutes and then washed with sterile water and then with 60% isopropanol. The differentiation was confirmed by staining lipid deposits in the cells with 5% (wt/vol) Oil Red O diluted in isopropanol.

For chondrogenic differentiation, 2% FBS, 1% P/S, 50 μg/mL ascorbate-2-phosphate, 100 μg/mL sodium pyruvate, 1% insulin-transferrin-selenium (ITS, GIBCO), 100 nM dexamethasone, 40 μg/mL L-proline and 10 ng/mL TGF-β3 (Prospec, East Brunswick, N.J., USA) were added to DMEM-high glucose. The differentiation medium was replaced every 2 days for 2 weeks. When the differentiation was completed, the cells were fixed with 4% PFA for 15 minutes and then washed with sterile water. The differentiation was confirmed by staining acidic mucopolysaccharides such as glycosaminoglycan with Alcian blue.

Inflammatory Cell Models

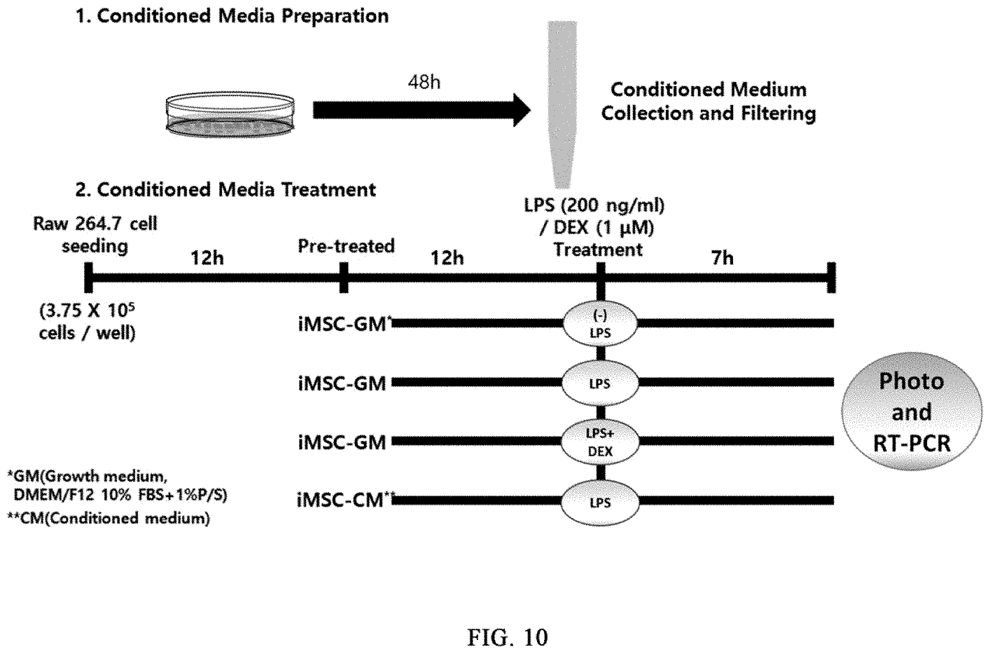

Raw 264.7 cells, which are macrophages used in inflammatory cell models, were used to evaluate the anti-inflammatory activity of the iPSC-MSCs of the present disclosure. The Raw 264.7 cells were cultured in an α-MEM (minimum essential medium) supplemented with 10% FBS and 1% P/S. First, after culturing the iPSC-MSCs in a culture vessel for 48 hours, the conditioned medium was collected, filtered through a 0.20-μm syringe filter and then kept in a refrigerator at 4° C. The Raw 264.7 cells were inoculated onto a 6-well culture dish at 3.75×10⁵ cells per well. 12 hours later, when the cells adhered to the bottom, the medium was replaced with the conditioned medium prepared above. 12 hours later, all the groups including the conditioned medium group, except for a control group, were treated with 200 ng/mL LPS (lipopolysaccharide, Sigma, USA) as shown in FIG. 10. A positive control groups were treated with LPS and 1 μM DEX (dexamethasone, Peprotech, USA). 7 hours later, images were recorded and total RNAs were isolated. Then, RT-PCR was conducted using the inflammatory marker IL-6.

Isolation of Total RNAs and RT-PCR

The total RNAs of the Raw 264.7 cells were extracted using a Labo Pass kit and TRIzol (Cosmo Genetech, Seoul, Korea) according to the manufacturer's instructions. The concentration of the total RNAs was measured using a Nanodrop (ND1000) spectrophotometer (Nanodrop Technologies Inc., Wilmington Del., USA). Then, cDNAs were synthesized using 2 μg of the total RNAs and M-MLV reverse transcriptase (Promega) according to the manufacturer's instructions. The RT-PCR reaction was analyzed on 2% agarose gel. The sequences of the used primers are given in Table 1:

TABLE 1
Sequences of primers used in RT-PCR

| Genes | Forward | Reverse |
|---|---|---|
| IL-6 | GTC CTT CCT ACC CCA | TAA CGC ACT AGG TTT GCC |
| | ATT TCC A | GA |
| GAPDH | CTC ACT CAA GAT TGT | GTC ATC ATA CTT GGC AGG |
| | CAG CA | TT |

### Experimental Result

Formation of Spheroids Using BAM System

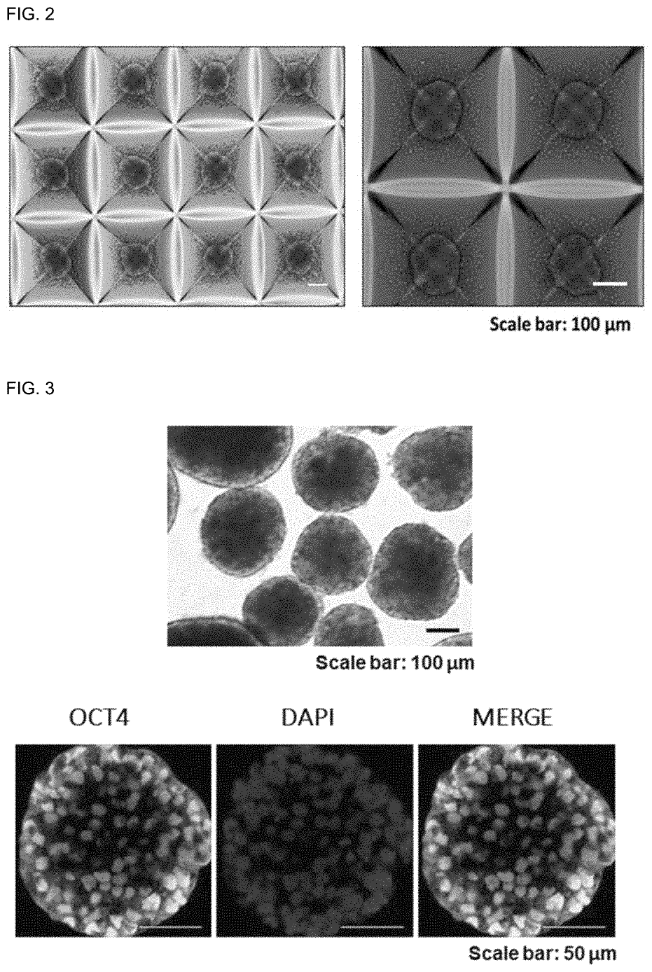

The iPSCs placed on the AggreWell plate were identified to form spherical EBs with homogenous morphology and size 24 hours later (FIG. 2).

Investigation of Pluripotency of Spheroids by Immunohistochemical Staining

The spheroids were stained green when stained with the pluripotency marker OCT4, and the nuclei were stained green and blue specifically when stained with DAPI. Therefore, it was confirmed that OCT4 is expressed in the nuclei and the spheroids derived from the iPSCs retain pluripotency (FIG. 3).

Production of iPSC-MSCs

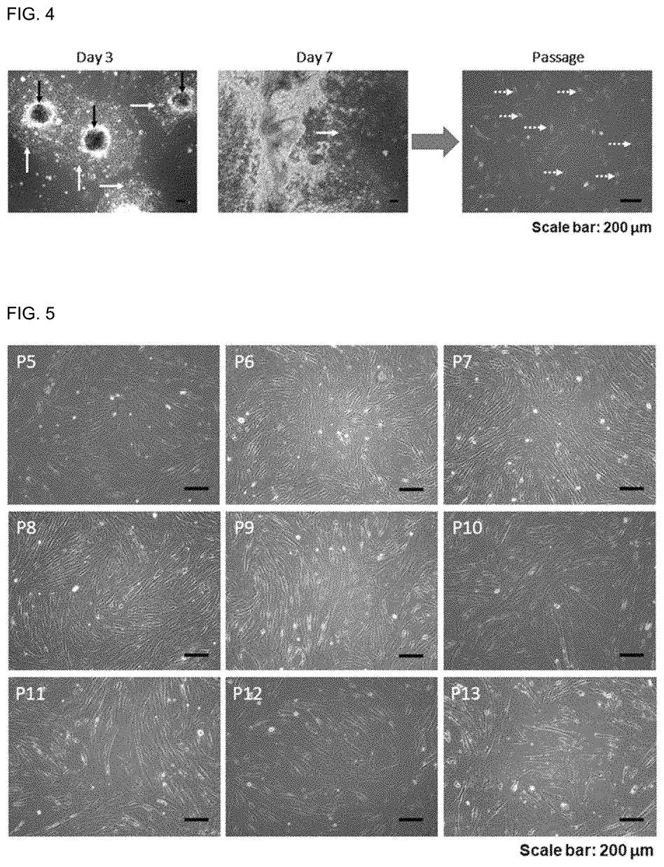

It was confirmed that the spheroids (black arrows) adhere to the bottom of the 0.1% gelatin-coated culture dish and cells protrude therefrom (white arrows). The protruding cells were increased with time. When the cells were passaged at 70-80%, the cells showed a uniform spindle shape (white broken arrows) as the passaging proceeded (FIG. 4).

Passaging of iPSC-MSCs and Cell Growth Curves

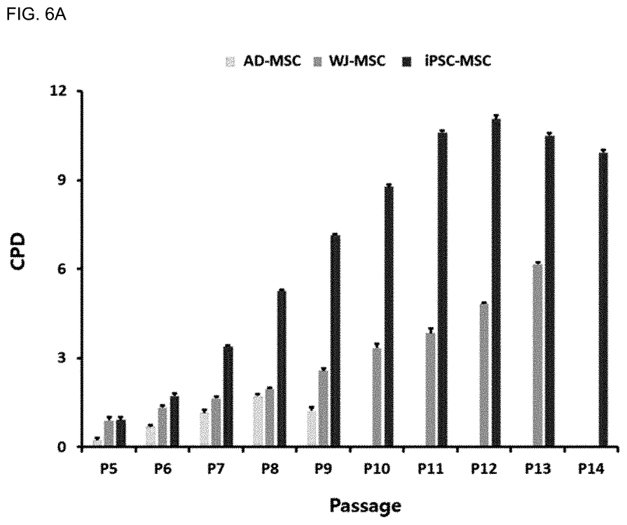

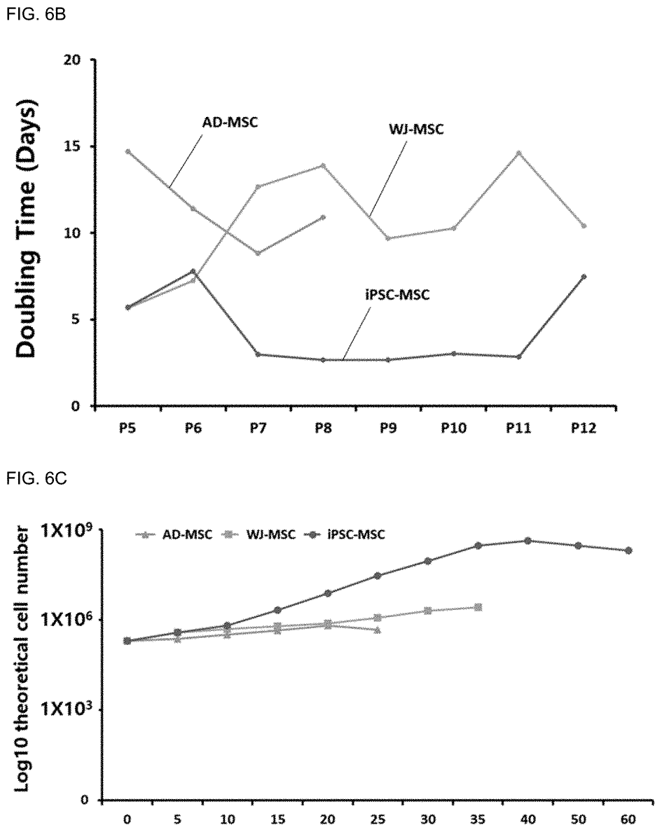

The iPSC-MSCs showed a spindle shape from P5 and could be passaged until P13 (FIG. 5). The cells were prepared into a stock solution and stored in LN₂. The cell growth curves were compared with hWJ-MSCs and AD-MSCs as control groups. As a result, the AD-MSCs showed increase in cell number up to P9 followed by decrease, whereas the hWJ-MSCs showed consistent increase even at P13. The iPSC-MSCs showed remarkably higher CPD and a larger number of cumulative cells than the control groups, and also showed a faster doubling time than the control groups (FIG. 6).

Immunophenotype Testing by Flow Cytometry Analysis (FACS)

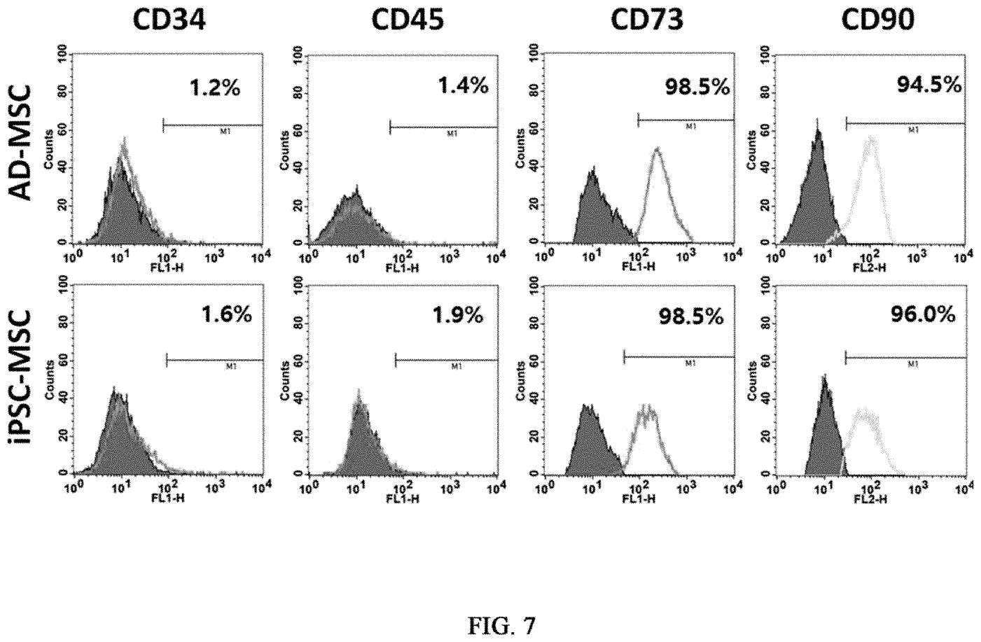

As a result of FACS analysis, the iPSC-MSCs were confirmed to be positive for anti-CD73 and anti-CD90 and negative for anti-CD34 and anti-CD45, unlike the control group AD-MSCs (FIG. 7). Through this, it was confirmed that the iPSC-MSCs produced using the BAM system have the distinct characteristics of mesenchymal stem cell.

Induction of Differentiation into Bone, Fat and Cartilage

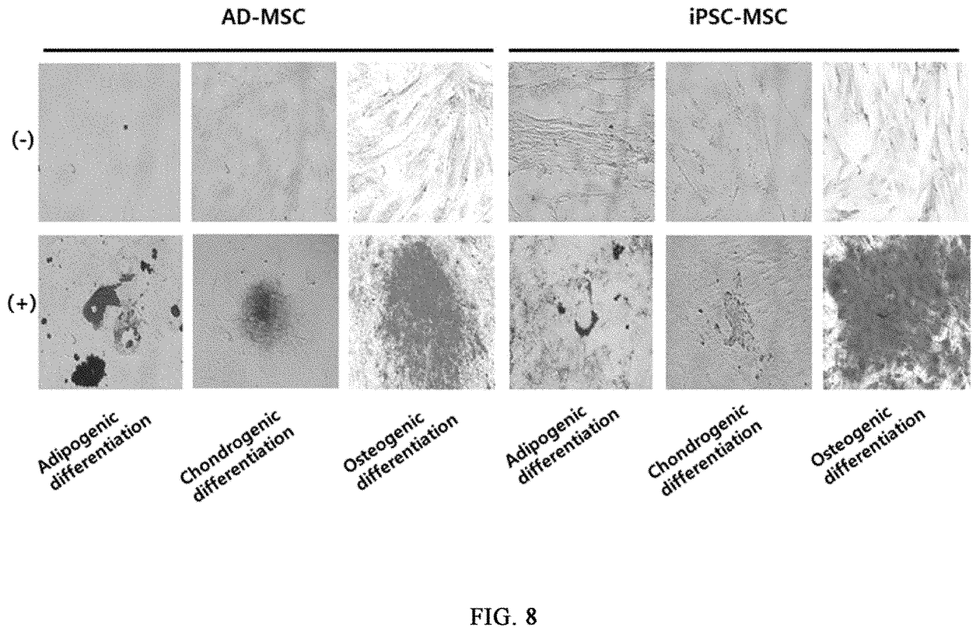

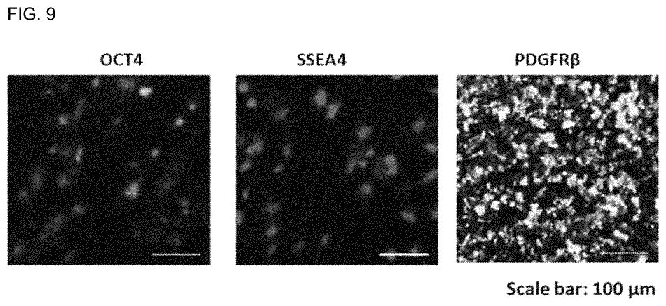

The AD-MSCs and iPSC-MSCs were induced to differentiate into bone, fat and cartilage. As a result of staining with Alizarin Red S, Oil Red O and Alcian blue after 2 weeks of differentiation, it was confirmed that the iPSC-MSCs were differentiated into bone, fat and cartilage similarly to the AD-MSCs used as a control group. Especially, bone formation was achieved effectively (FIG. 8).

Identification of iPSC-MSCs as Mesenchymal Stem Cells by Immunohistochemical Staining

The iPSC-MSCs were subjected to ICC using the pluripotency markers OCT4 and SSEA4 and the mesenchymal stem cell marker PDGFRβ. The iPSC-MSCs showed a small number of the cells stained green with the OCT4 and SSEA4 markers but a large number of the cells stained green with the PDFGRβ marker. Through this, it was confirmed that the iPSC-MSCs lost pluripotency and were converted to mesenchymal stem cells.

Inflammatory Cell Models

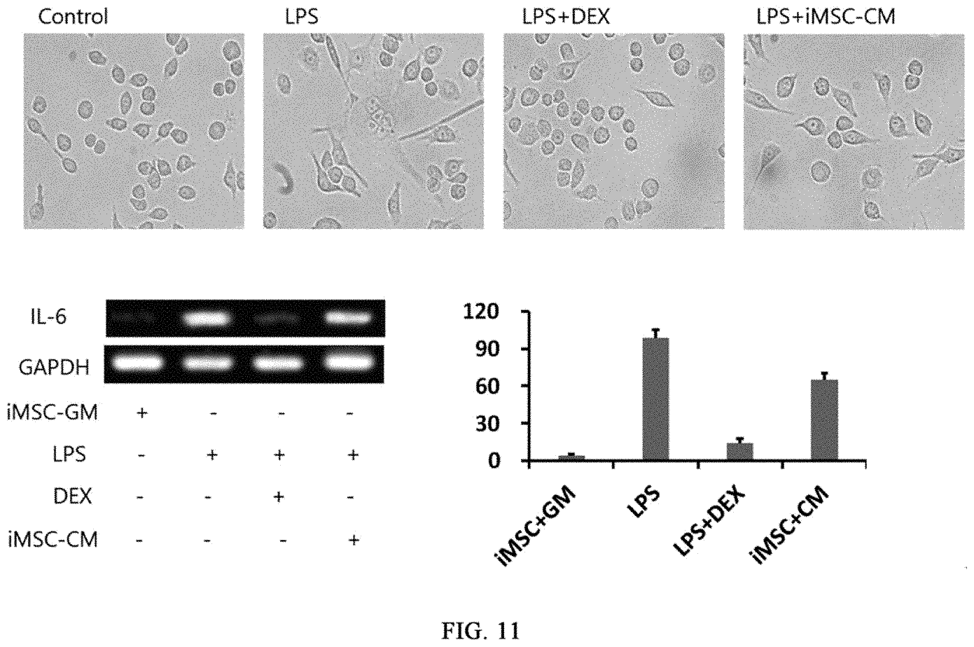

Round cells were observed in a group not treated with LPS, whereas large multi-nucleated cells were observed in the group in which inflammation was induced by treating with LPS. Meanwhile, multi-nucleated cells were not observed in the positive control group treated with LPS and DEX, and the large multi-nucleated cells were also nonexistent in the group treated with the conditioned medium of the iPSC-MSCs. As a result of investigating the expression of IL-6 by RT-PCR, the expression level was high as 98% in the LPS-treated group and low as 14% in the group treated with LPS and DEX. The expression level was 65% in the group treated with the conditioned medium of the iPSC-MSCs. Accordingly, it was confirmed that IL-6 is expressed at a lower level in the group treated with the conditioned medium of the iPSC-MSCs than the group treated with LPS (FIG. 11)

While specific exemplary embodiments of the present disclosure have been described in detail, it will be obvious to those having ordinary knowledge in the art that they are mere specific exemplary embodiments and the scope of the present disclosure is not limited by them. It is to be understood that the substantial scope of the present disclosure is defined by the appended claims and their equivalents.

We claim:

1. A method for producing mesenchymal stem cells from pluripotent stem cells, comprising:
(a) a step of forming embryoid bodies (EBs) by culturing pluripotent stem cells isolated from a subject;
(b) a step of forming spheroids by three-dimensionally culturing the embryoid bodies in a bioreactor under microgravity; and
(c) a step of differentiating the spheroids into mesenchymal stem cells by adherent-culturing in a culture vessel coated with an adhesive polymer.

2. The method according to claim 1, wherein the pluripotent stem cells are embryonic stem cells (ESCs) or induced pluripotent stem cells (iPSCs).

3. The method according to claim 1, wherein the pluripotent stem cells are induced pluripotent stem cells.

4. The method according to claim 1, wherein the step (a) is performed by three-dimensionally culturing the pluripotent stem cells in a multi-well culture plate.

5. The method according to claim 4, wherein the step (a) further includes a step of inducing cell aggregation during the three-dimensional culture through centrifugation.

6. The method according to claim 1, wherein the microgravity in the step (b) is induced by a microgravity simulator which offsets the gravity applied to the bioreactor by rotating the bioreactor.

7. The method according to claim 6, wherein the step (b) is performed by culturing the embryoid bodies for 3-8 days while rotating the microgravity simulator at 40-80 rpm.

8. The method according to claim 7, wherein the step (b) is performed by rotating the microgravity simulator first at 40-60 rpm and increasing the rotation speed by 5 rpm every day.

9. The method according to claim 1, wherein the adhesive polymer is selected from a group consisting of hyaluronic acid, alginate, heparin, fucoidan, cellulose, dextran, chitosan, albumin, fibrin, collagen and gelatin.

10. The method according to claim 9, wherein the adhesive polymer is gelatin.

11. Mesenchymal stem cells produced by the method according to claim 1.

12. A composition for treating a bone or cartilage disease, comprising the mesenchymal stem cells according to claim 11 as an active ingredient.

13. A composition for treating an inflammatory or autoimmune disease, comprising the mesenchymal stem cells according to claim 11 as an active ingredient.

14. The composition according to claim 13, wherein the inflammatory or autoimmune disease is rheumatoid arthritis, reactive arthritis, type 1 diabetes, type 2 diabetes, systemic lupus erythematosus, multiple sclerosis, cryptogenic fibrosing alveolitis, polymyositis, dermatomyositis, localized scleroderma, systemic scleroderma, colitis, inflammatory bowel disease, Sjorgen's syndrome, Raynaud's phenomenon, Bechet's disease, Kawasaki's disease, primary biliary sclerosis, primary sclerosing cholangitis, ulcerative colitis, graft-versus-host disease (GVHD) or Crohn's disease.
